(19) United States
(12) Patent Application Publication
(10) Pub. No.: US 2010/0174304 A1
(43) Pub. Date: Jul. 8, 2010

(54) Methods And Devices For Creating Electrical Block At Specific Targeted Sites In Cardiac Tissue

(76) Inventors: Robert S. Schwartz, Rochester, MN (US); Richard Cornelius, Wayzata, MN (US); William Swanson, St. Paul, MN (US)

Correspondence Address: INSKEEP INTELLECTUAL PROPERTY GROUP, INC, 2281 W. 190TH STREET, SUITE 200, TORRANCE, CA, 90504, US

(21) Appl. No.: 12/726,304

(22) Filed: Mar. 17, 2010

Related U.S. Application Data
(63) Continuation of application No. 11/670,148, filed on Feb. 1, 2007, now abandoned.

(51) Int. Cl. A61B 17/32

(52) U.S. Cl. 606/167

(57) ABSTRACT

The present invention provides a mechanical injury device having cutting elements for injuring tissue and thereby creating electrical block that can prevent atrial fibrillation. These cutting elements may preferably be removable, breakaway, or simply integral to the injury device and may be delivered, for example, by catheter or hand tool.

### CROSS REFERENCE TO RELATED APPLICATIONS

This application claims the benefit of U.S. Provisional Application 60/467,298, entitled Improved Methods And Devices For Creating Electrical Block At Specific Targeted Sites In Cardiac Tissue, filed May 1, 2003, the entire contents of which are hereby incorporated by reference.

### BACKGROUND OF THE INVENTION

Pumping of the human heart is caused by precisely timed cycles of compartmental contractions of the heart muscle which lead to an efficient movement of blood into the heart and out to the various bodily organs and back again to the heart. These precisely timed cycles are controlled and directed by electrical signals that are conducted through the cardiac tissue and can be referred to as pacing signals.

The sinoatrial node (SA node) is the heart's natural pacemaker, located in the upper wall of the right atrium. The SA node spontaneously depolarizes and generates electrical impulses that travel throughout the heart wall causing both the left and right atria to sequentially contract according to a normal rhythm for pumping of the heart. These electrical impulses continue to the atrioventricular node (AV node) and down a group of specialized fibers called the His-Purkinje system to the ventricles. This electrical pathway must be exactly followed for proper functioning of the heart.

When the normal sequence of electrical impulses changes or is disrupted, the heart rhythm often becomes abnormal. This condition is generally referred to as an arrhythmia and can take the form of such arrhythmias as tachycardias (abnormally fast heart rate), bradycardias (abnormally slow heart rate) and fibrillations (irregular and typically quite rapid cardiac electrical activity).

Of these abnormal heart rhythms, fibrillation, and particularly atrial fibrillation, is gaining attention by clinicians and health workers. Atrial fibrillation develops when a disturbance in the electrical signals causes the two upper atrial chambers of the heart to quiver instead of function as a synchronized pump. When this happens, blood is not efficiently pumped from the atrial chambers, thus creating a situation where the blood may pool and even clot inside the atria. Such clotting can be very serious insofar as the clot can, for example, leave the atrial chamber and block an artery in the brain or coronary artery, and thereby cause a stroke or heart attack in the individual.

A variety of treatments have been developed over the years to treat atrial fibrillation, namely, treatments to either mitigate or eliminate electrical conduction pathways that lead to the arrhythmia. Those treatments include medication, electrical stimulation, surgical procedures and ablation techniques. In this regard, typical pharmacological treatments have been previously disclosed in U.S. Pat. No. 4,673,563 to Berne et al.; U.S. Pat. No. 4,569,801 to Molloy et al.; and also by Hindricks, et al. in “Current Management of Arrhythmias” (1991), the contents of which are herein incorporated by reference.

Surgical procedures, such as the “maze procedure”, have also been proposed as alternative treatment methods. The “maze” procedure attempts to relieve atrial arrhythmias by restoring effective atrial systole and sinus node control through a series of incisions.

The maze procedure is an open heart surgical procedure in which incisions are made in both the left and right atrial walls which surround the pulmonary vein ostia and which leave a “maze-like” pathway between the sino-atrial node and the atrio-ventricular node. The incisions are sewn back together but result in a scar line which acts as a barrier to electrical conduction.

Although the “maze” procedure has its advantages, in practice it can be complicated and a particularly risky procedure to perform since the surgeon is making numerous physical incisions in the heart tissue. Due in part to the risky nature of the maze procedure, alternative, catheter-based treatments have been advanced. Many of these catheter devices create the desired electrical block using ablation devices designed to scarred lesions by burning, freezing, or other noxious methods directed at target tissue. Examples of these devices can be seen in U.S. patents: U.S. Pat. No. 6,254,599 to Lesh; U.S. Pat. No. 5,617,854 to Munsif; U.S. Pat. No. 4,898,591 to Jang et al.; U.S. Pat. No. 5,487,385 to Avitall; and U.S. Pat. No. 5,582,609 to Swanson, all incorporated herein by reference.

Although ablation catheter procedures remain less invasive than previous surgical methods like the “maze” procedure, they nevertheless retain a significant element of risk. For example, ablation procedures often utilize high power RF energy or ultrasonic energy, which may adequately create electrical block, but their inherent destructive nature allows for the possibility of unintended damage to the target tissue or nearby areas.

More recently, implantable devices have been used near or within the pulmonary vein to cause electrical block, as seen in the pending and commonly owned U.S. patent application Ser. No. 10/192402 entitled Anti-Arrhythmia Devices And Methods Of Use, filed Jul. 8, 2002, the contents of which are incorporated by reference. Once implanted, these devices cause injury to target tissue near the ostium of the pulmonary vein but often do not create an acute electrical block. Rather, the electrical block may develop as the healing process runs its course on the injury. Other examples of such devices are seen in the pending commonly owned U.S. patent application Ser. No. 10/792,111 entitled Electrical Block Positioning Devices And Methods Of Use Therefor, filed Mar. 2, 2004, the contents of which are hereby incorporated by reference.

However, controlling the injury caused by the implant device can remain difficult since these techniques often require the implant device to remain in the patient permanently. Further, it can be difficult for an implant device to securely fit at a desired position within a patient, especially near the ostium of a pulmonary vein. What is needed is a device that can create controlled damage such as is caused by a permanent implant but without the drawbacks of a permanent implant.

### OBJECTS AND SUMMARY OF THE INVENTION

It is an object of the present invention to provide an easily controlled mechanical injury device to create electrical block within an atrium or pulmonary venous region of a patient.

It is another object of the present invention to provide a mechanical injury device that reliably creates lines of electrical block in an atrial or pulmonary vein region of a patient.

It is a further object of the present invention to overcome the limitations of the prior art.

The present invention achieves these objectives by providing a mechanical injury device having cutting elements for injuring tissue in the patient and thereby creating electrical block. These cutting elements may be removable, breakaway, or simply integral to the injury device and may be delivered, for example, by a catheter or hand tool.

### BRIEF DESCRIPTION OF THE DRAWINGS

FIG. 1 illustrates a side view of a forward injury arm catheter according to the present invention;

FIG. 2 illustrates a side view of a reverse injury arm catheter according to the present invention;

FIG. 3 illustrates a side view of a bent injury arm catheter according to the present invention;

FIG. 4A illustrates a perspective view of a roller head according to the present invention;

FIG. 4B illustrates a perspective view of a cutting element according to the present invention;

FIG. 4C illustrates a perspective view of a cutting element according to the present invention;

FIG. 5 illustrates a perspective view of a roller head delivery assembly according to the present invention.

FIG. 6 illustrates a top view of a flattened roller head according to the present invention;

FIG. 7 illustrates a top view of a flattened roller head according to the present invention;

FIG. 8 illustrates a top view of a flattened roller head according to the present invention;

FIGS. 9A-9F illustrate various views of a removable cutting element according to the present invention;

FIGS. 10A-10D illustrate various views of a breakaway cutting element according to the present invention;

FIGS. 11A-11B illustrate various views of a hand-held injury device according to the present invention;

FIG. 12 illustrates a perspective view of a hand-held injury device according to the present invention;

FIG. 13 illustrates a side view of an expandable mesh injury device according to the present invention;

FIG. 14 illustrates a side view of a cutting element deployment catheter according to the present invention;

FIG. 15 illustrates a side view of the deployment arm illustrated in FIG. 14; and

FIG. 16 illustrates a side view of the hub for the cutting element deployment catheter illustrated in FIGS. 14 and 15.

### DETAILED DESCRIPTION OF THE INVENTION

The present invention contemplates the use of cutting elements such as needle or pin shapes to cause injury to desired target tissue. The target tissue is typically the atrial tissue surrounding the ostia of a pulmonary vein, however, it can also include tissue inside the ostia or tissue inside the pulmonary vein downstream of the ostia. The injury results in scarring of the target tissue and the scarred tissue results in the formation of a conduction block that prevents the aberrant signals from causing the atrial fibrillation. One method of efficacy may be to introduce hemorrhage within the wall of the target tissue that typically heals with a non-electrically active scar. As described below, the cutting elements may preferably be integral with the device, allowing for one-time injury, or the cutting elements may also preferably be removable or breakaway, allowing for prolonged tissue damage.

As described elsewhere in this application, these cutting elements may be preferably deployed with a variety of different devices, such as a roller head on a catheter or hand held tool, an expandable catheter, or by way of a deployment tube. Thus, a user is better able to create a controlled, desired injury to a patient, resulting in a potentially safer procedure and the formation of a more precise electrical conduction block.

### Injury Arm Catheter

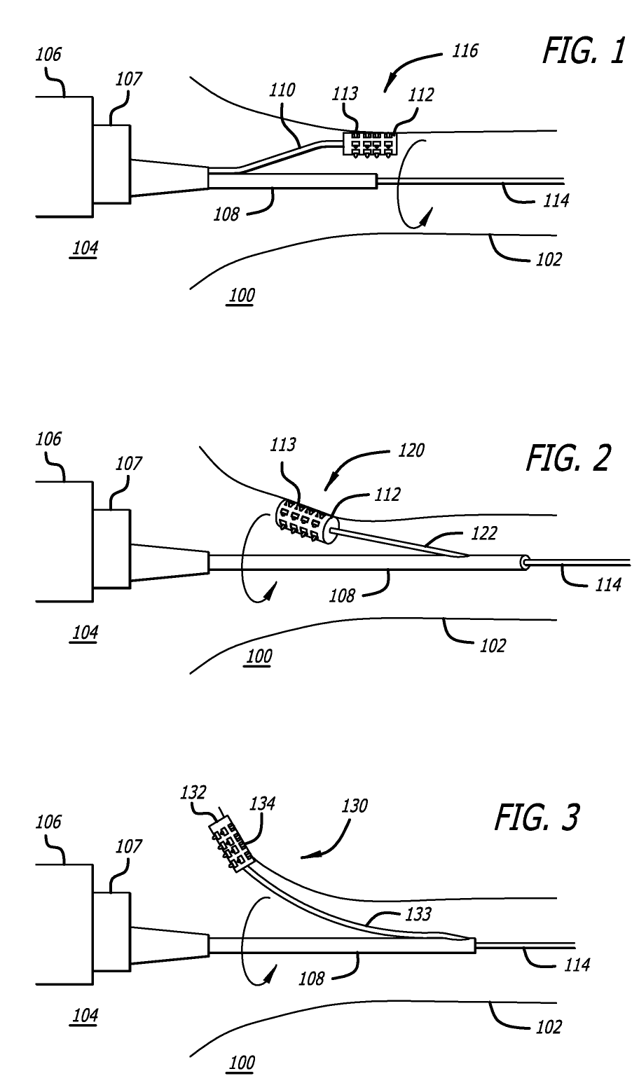

FIG. 1 illustrates a preferred embodiment of a forward injury arm catheter 116 according to the present invention as deployed in a pulmonary vein 102. The forward injury arm catheter 116 has a forward injury arm 110 with a roller head 112 fixed to a catheter body 108. Disposed on the roller head 112 are cutting elements 113 which may be directed to cause injury at a desired target site.

The catheter body 108 has an inner lumen (not shown) sized for a guide wire 114 which may assist a user in positioning the forward injury arm catheter 116 at a desired location, e.g. in a pulmonary vein 102 or pulmonary vein ostial opening 100. Near the distal end of the catheter body 108 is forward injury arm 110 which, at one end, is fixed to the catheter body 108 and extends radially and distally away from the catheter body 108 when deployed. The forward injury arm 110 is preferably preset to expand radially away from the catheter body 108, to a position similar of that seen in FIG. 1.

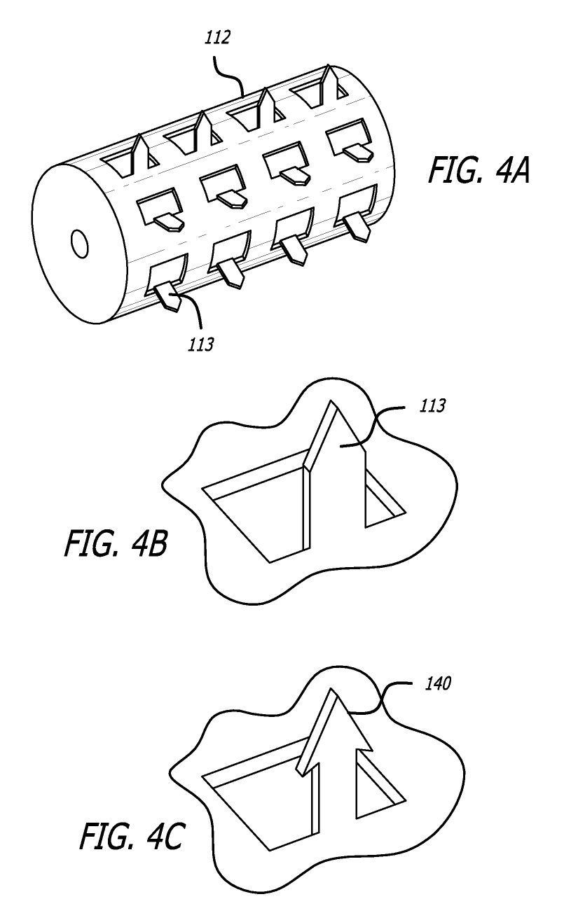

The roller head 112 is coupled to the distal end of forward injury arm 110 so as to freely axially rotate. As best seen in FIGS. 4A and 4B, pin shaped cutting elements 113 located around the circumference of the roller head 112 are preferably angled perpendicularly away from the roller head 112. Preferably, the roller head 112 may be formed from a small section of hypotube or a similar tube shape composed of a rigid metal or plastic material. A desired pin pattern may be laser cut into the perimeter of the tube and the cutting elements 113 can be formed to project out from the surface of the tube. A variety of different shapes and patterns of cutting element 113 may be used on the roller head 112, examples of which are discussed elsewhere in this application.

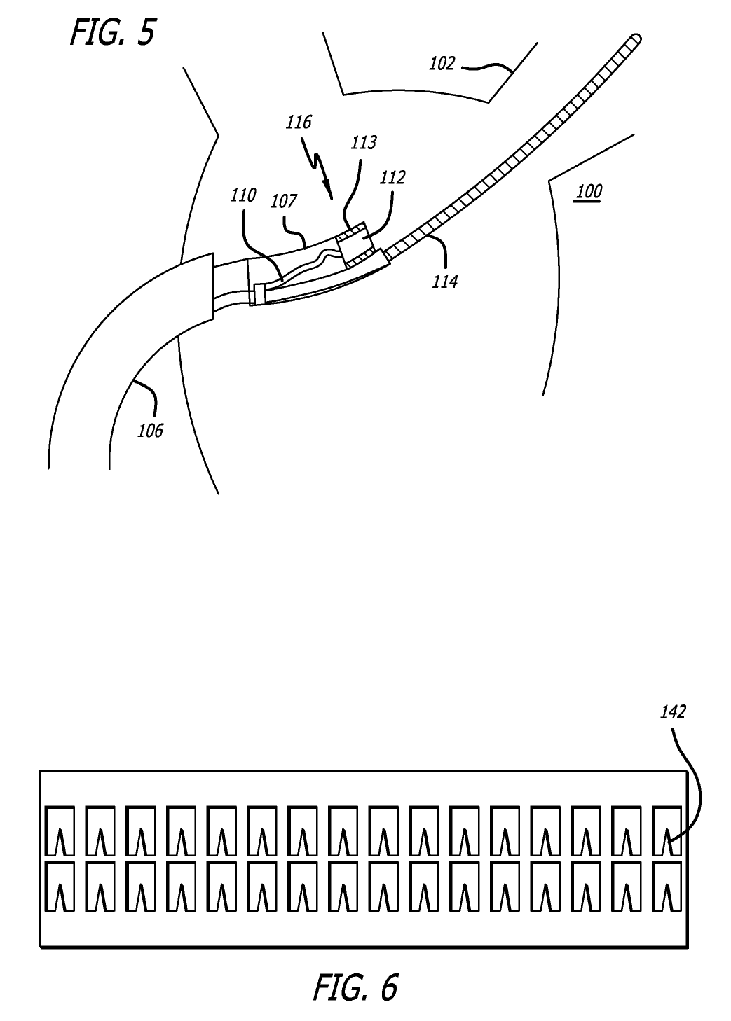

In operation, the guide wire 114 is inserted within a patient's vessel and positioned at a desired target location, for example, the guide wire 114 may be transeptally positioned within a pulmonary vein 102 of a left atrium 104. The catheter body 108, the forward injury arm 110 and the roller head 112 are packed within the transeptal sheath 106 to reduce unintended injury to non-target areas of the patients vessels. This can be accomplished with a thin-walled sleeve 107, seen best in FIG. 5, which holds the roller head 112 down to a compressed diameter for passage through the transeptal sheath 106 and the atrium. This sleeve 107 also shields the cutting elements 113 from damaging the transeptal sheath 106 in transit. The sleeve 107 is retractable to release the roller head 112 from its compressed diameter when ready to advance the roller head 112 into position at the targeted treatment site. Next, forward injury arm catheter 116 is advanced along the guide wire 114 to a desired target location, e.g. a pulmonary vein 102 or the ostial opening 100 of a pulmonary vein. The sleeve 107 is pulled back, uncovering a portion of the catheter body 108 and forward injury arm 110. The forward injury arm 110 expands away from the catheter body 108 until the roller head 112 contacts the target tissue, causing cutting elements 113 to create points of injury. The catheter body 108 is then rotated, which causes the forward injury arm 110 and the roller head 112 to move in a circular path around the inside of pulmonary vein 102. The roller head 112 itself rotates axially, reducing resistance and facilitating the overall rotational movement of the catheter body 108 and forward injury arm 110. Thus, the injury elements 113 on the roller head 112 may cause a continuous, circular line of electrical block as the injury heals and forms scar tissue. The forward injury arm 110 may be repositioned to repeat the injury in other locations to achieve a desired electrical block. When the user is finished, the roller head 112 can be compressed back within the sleeve 107 after completing treatment by advancing the sleeve forward. This can be facilitated by the shape of the injury arm 110 and by angling the first row of cutting elements 113 as shown in FIG. 5. The forward injury arm catheter 116 can then be removed through the transeptal sheath 106. In a preferred embodiment the roller head 112 and cutting elements 113 will have a diameter of about 0.100 inches or less to allow it to be compressed down against the central catheter lumen and still allow it to be sleeved and fit inside a 10-11 French sheath.

FIG. 2 illustrates a preferred embodiment of a reverse injury arm catheter 120, having an overall similar design to the previous embodiment. However, the reverse injury arm catheter 120 has a reverse injury arm 122 fixed to a distal end of the catheter body 108 and extends in a proximal direction (an opposite direction to the preferred embodiment of FIG. 1). The reverse injury arm 122 is preset to move away from the catheter body 108 when in a deployed state, pressing the roller head 112 against the inner surface of pulmonary vein 102. The reverse injury arm catheter 120 may include a tether wire (not shown) having one end fixed to the reverse injury arm 122 and passing into a lumen (not shown) within the catheter body 108. With this tether wire, a user may move the reverse injury arm 122 close to the catheter body 108, allowing the transeptal sheath 106 to be slid over both the reverse injury arm 122 and the remaining exposed portion of the catheter body 106.

The reverse injury arm catheter operates in a manner similar to the previous embodiment of FIG. 1, namely the guide wire 114 is initially positioned at a target location, followed by the transeptal sheath 106 containing the catheter body 108, the reverse injury arm 122 and roller head 112. Once in position, the transeptal sheath 106 is moved proximally to expose the reverse injury arm 122 and roller head 112. Once deployed, the reverse injury arm 122 moves outward from the catheter body 108, axially, until the roller head 112 contacts the target area, e.g. the inside of the pulmonary vein 102 or the ostial opening 100 of the pulmonary vein 102. The catheter body 108 is rotated by the user, moving the reverse injury arm 122 and roller head 112 around the ostium 100 in a circular path. After at least one complete rotation, the cutting elements 113 have formed a continuous circular line of injury which gradually creates a line of electrical block as a result of forming scar tissue in the healing process.

FIG. 3 illustrates a preferred embodiment of a bent injury arm catheter 130, generally similar to the preferred embodiment of FIG. 2. However, the bent injury arm catheter 130 differs in that it has an injury arm 122 with a preset curve and a roller head 132 with an overall rounded shape.

The injury arm 133 may be formed with varying preset bends, depending on the desired target area. For example, the injury arm 133 of FIG. 3 illustrates a bend appropriate to reach the ostium 100 of a pulmonary vein 102 when deployed. The roller head 132 has an overall rounded shape with cutting elements 134 disposed upon the surface. This injury arm 133 and roller head 132 combination allow the bent injury arm catheter 133 to create continuous lines of injury in locations otherwise perhaps hard to achieve by the preferred embodiments illustrated in FIGS. 1 and 2.

### Cutting Elements

The cutting elements described in this application may take a variety of shapes and patterns, as seen in the preferred embodiments of FIGS. 4A-10D. Cutting elements may be configured to cause varying levels of tissue damage, for example, or to create multiple lines of injury with varying length, width, and spacing. It is desirable to create local bleeding into the tissue wall without creating significant bleeding through the wall. In one preferred embodiment of the cutting elements used for the pulmonary vein 102 may be about 0.050 inches in length, about 0.015 inches in width and about 0.015 inches in thickness. Further, theses cutting elements may be composed from a wide range of possible materials, such as metals, engineering polymers, biodegradable polymers, or drug eluting polymers, depending on the needs of the user.

FIGS. 4B and 4C illustrate examples of two preferred embodiments of cutting elements 113 and 140. Cutting element 113 has an elongated pin shape while cutting element 150 includes two side barbs. These shapes may be further modified by, for example, varying the cutting element thickness, width, length, profile shape, and composition.

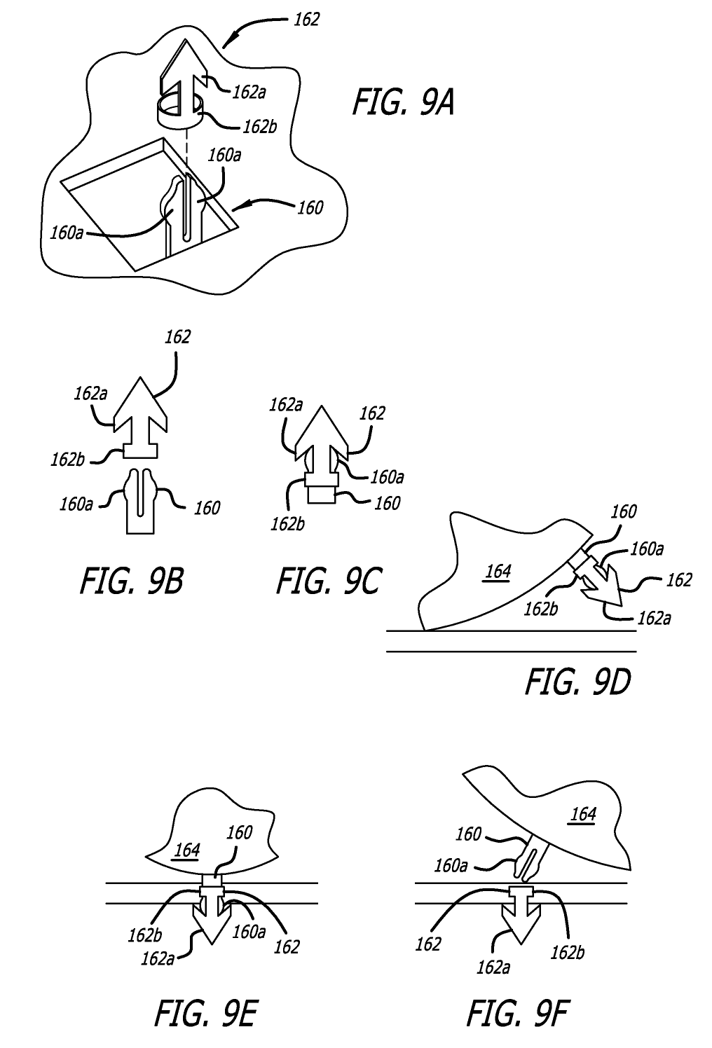

FIGS. 9A-9F illustrate a further preferred embodiment of removable cutting element 162 according to the present invention wherein the cutting elements 162 remain fixated in the target tissue and thereby create additional injury at the target site. The removable cutting element 162 has a sharp, barbed point 162a at one end and a locking ring 162b at the other. The cutting element post 160 consists of an upwardly positioned post having two prongs, each of which has a protrusion 160a. The locking ring 162b of the removable cutting element 162 slides onto cutting element post 160, past the protrusions 160a, and locking in place as seen best in FIG. 9C.

FIGS. 9D-9F illustrate the removable cutting element 162 in operation on a roller head 164. The removable cutting element 162 is initially locked onto cutting element post 160 which is then directed into an area of target tissue by roller head 164 rolling over the target tissue. The removable cutting element 162 penetrates the target tissue by the rolling force of roller head 164. As the roller head 164 rolls away from the penetration point, the barbs 162a hold the cutting element 162 within the tissue, allowing the cutting element post 160 to pull out of locking ring 162b, leaving the cutting element 162 in the target tissue.

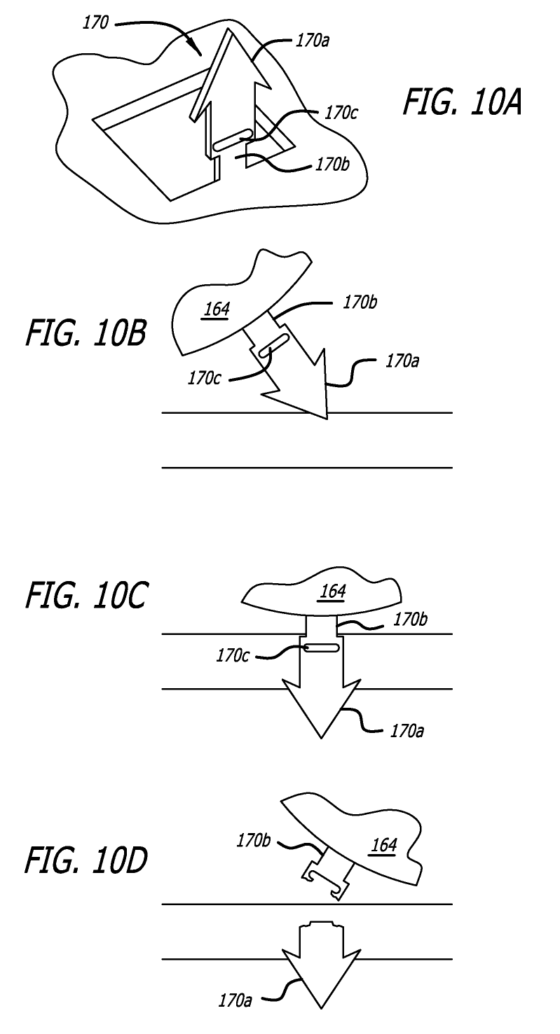

FIGS. 10A-10D illustrate yet another preferred embodiment of a breakaway cutting element 170 according to the present invention which breaks off during a procedure within a target tissue to create further injury. The breakaway cutting elements 170 are composed of a base 170b and a breakaway barbed tip 170a. An aperture 170c is located between the base 170b and the barbed tip 170a to encourage the barbed tip 170a to break off of the base 170b when placed in tension.

FIGS. 10B-10D illustrate the breakaway cutting elements 170 in operation as part of roller head 164. The roller head 164 rolls over a target tissue, forcing the barbed tip 170a into the tissue. As the roller head continues rolling, base 170b pulls against the anchoring force of the barbed tip 170a, and further breaks away from the barbed tip 170a. Thus, the barbed tip 170a is left within the target tissue to cause a desired amount of damage and consequently causing electrical block.

FIGS. 6-8 illustrates various preferred embodiments of example cutting element patterns. These figures illustrate example roller heads in a “flattened” view with patterns created with cutting lasers, chemical etching or similar fabrication techniques.

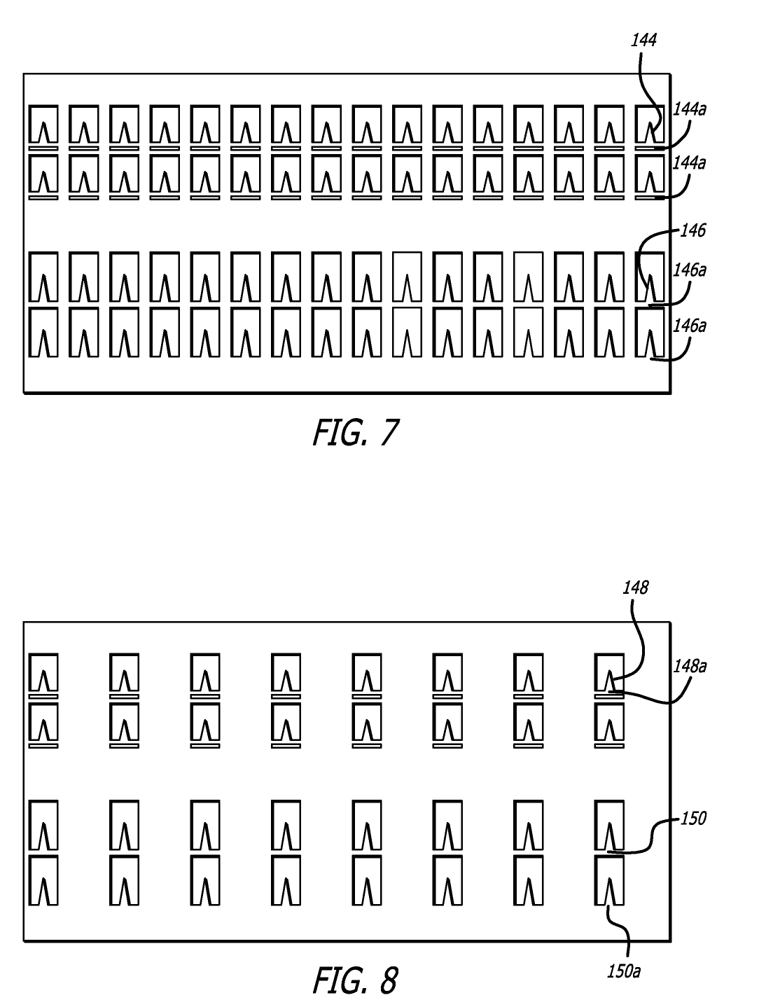

Looking first to a preferred embodiment illustrated in FIG. 6, cutting elements 142 are elongated needle shapes arranged in two closely positioned rows. FIG. 7 illustrates a dual row variation according to the present invention with one set of cutting elements 146 formed by bending the base 146a of the cutting element 146 and one set of elements 144 formed up by twisting the bar 144a of material at the base of the cutting element 144. FIG. 8 shows the same types of cutting elements as shown in FIG. 7, having a row of cutting elements 150 formed by bending the base 150a and a row of cutting elements 148 formed by twisting the bar 148a of material at the cutting element 148 base., both of which are less densely spaced than the rows of FIG. 7.

### Hand Held Injury Device

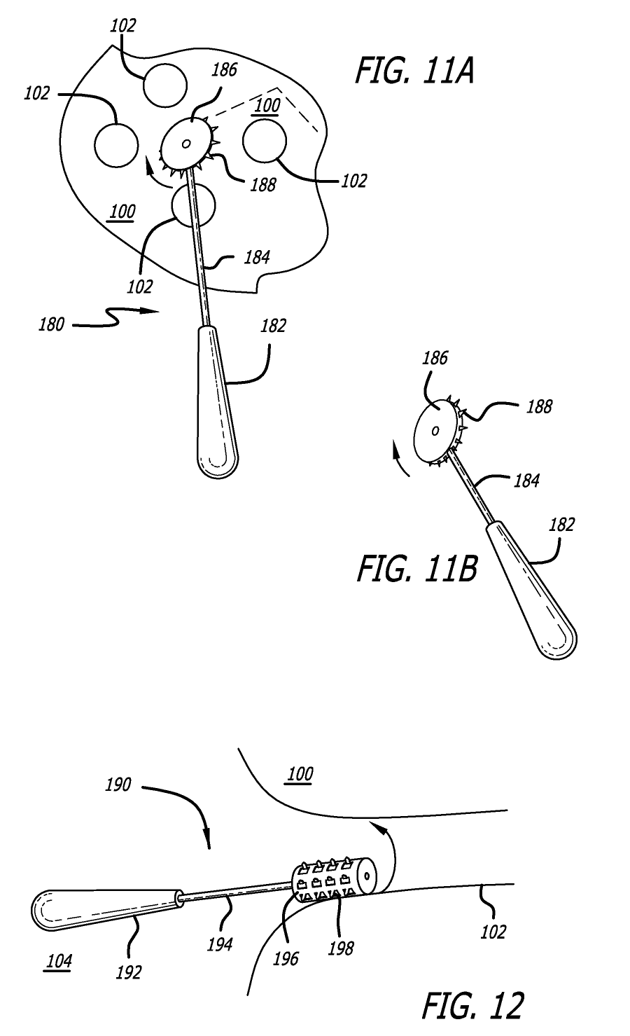

Turning now to FIGS. 11A and 11B, a preferred embodiment of a hand-held injury device 180 is illustrated according to the present invention, including a roller head 186 attached to a handle 182. This hand-held injury device 180 allows a user to create injury to a patient at an ostial opening 100 of a pulmonary vein 102 of the left atrium 104 as shown in FIG. 11A during surgical procedures that expose a desired target tissue, e.g. a mitral valve repair procedure. Alternatively, the hand-held injury device 180 may also be used during procedures where the left atrium is not open, e.g. in connection with coronary artery bypass graft (CABG) procedure. In this case the device would be used on the epicardial surface of the heart.

The roller head 186 has cutting elements 188 disposed along the outer diameter of its surface and is further rotationally mounted to arm 184. At the opposite end of arm 184 is handle 182.

In operation, a user grasps the handle 182 and directs the roller head 186 to the target tissue area (e.g. ostium 100 of the pulmonary vein 102) and rolls a continuous line where electrical block is desired. In this respect, the hand-held injury device 180 functions in a similar fashion to a pizza cutter, allowing for a narrow band of injury.

FIG. 12 illustrates yet another preferred embodiment of a hand-held injury device 109 according to the present invention. A cylinder roller head 196 similar to the embodiment of FIG. 4A is rotatably mounted to arm 194 with a handle 192. The outer diameter surface of cylinder roller head 196 is disposed with cutting elements 198, allowing for a larger injury area compared to the preferred embodiment of FIG. 11A.

To operate, a user simply grasps the handle 192 and positions the roller head 196 against the desired target area (e.g. the ostium 100 of the pulmonary vein 102), pressing the cutting elements 198 into the tissue to create a line of injury that results in an electrical block.

### Expandable Mesh Injury Catheter

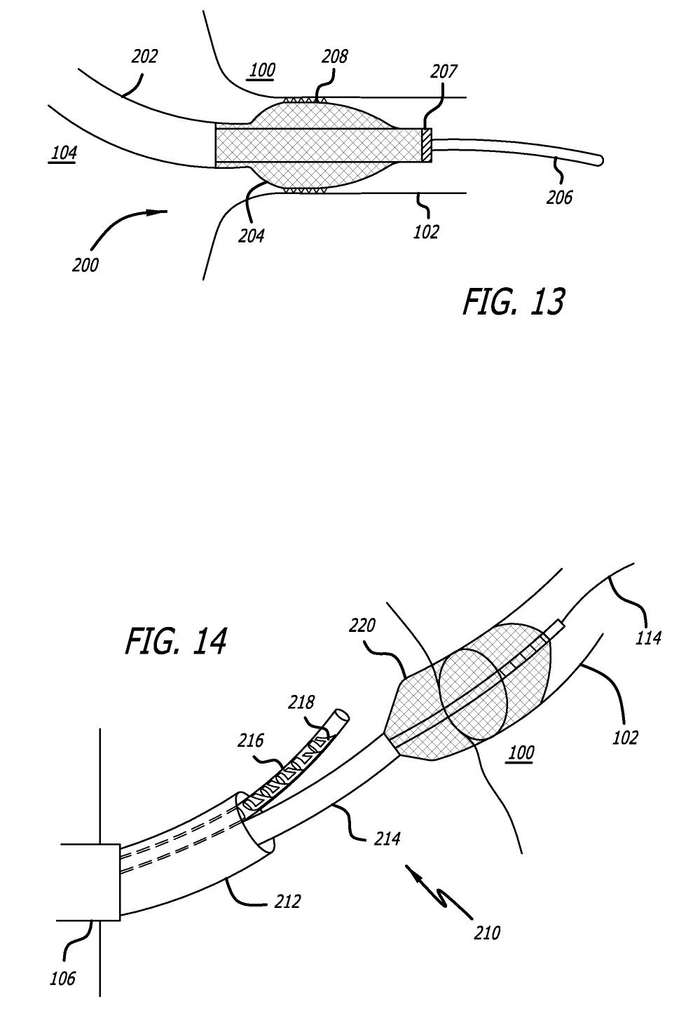

FIG. 13 depicts yet another preferred embodiment of a mesh injury catheter 200 according to the present invention, including cutting elements 208 fixed to the outer circumference of an expandable mesh section 204. Like many prior art catheters, the present preferred embodiment includes a guide wire 206 that may be positioned through an inner lumen of catheter body 202, allowing the guide wire 206 to be advanced to a desired target location within a patient (e.g. within a pulmonary vein 102), followed by the catheter body 202.

The expandable mesh section 204 is composed of elongated elements, preferably metal, woven together into a mesh. The distal end 207 of mesh section 204 is connected to a control cable within the catheter body 202 and is not connected to the catheter body 202. Thus, when a user pulls the control cable, the distal end 207 of expandable mesh section 204 moves in a proximal direction, expanding the mesh section 204 against the surrounding tissue. Since the cutting elements 208 are located on the outer surface of the expandable mesh section 2-4, the cutting elements 208 are pushed into the surrounding tissue, causing injury. In this manner, a user may position the distal end of the mesh injury catheter 200 at a desired location (a pulmonary vein 102 of a left ventricle, for example) to cause damage and ultimately a continuous line of electrical block.

### Cutting Element Deployment Catheter Arm

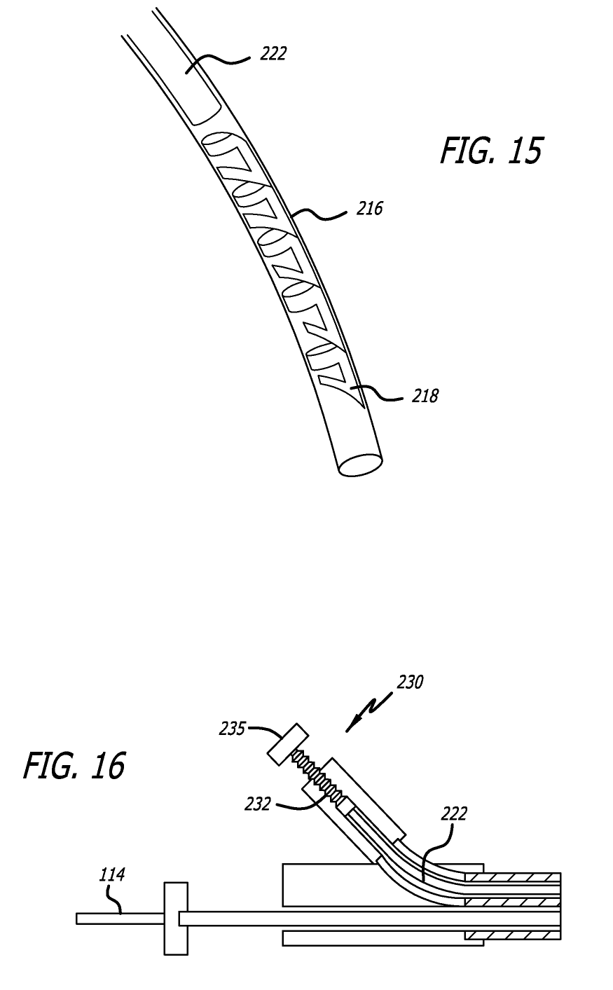

Referring to FIGS. 14 and 15, a preferred embodiment of a cutting element deployment catheter 210 can be seen according to the present invention. The cutting element deployment catheter 210 contains a cutting element deployment arm 216, seen best in FIG. 15, that may be positioned at a desired position within a patient to deploy cutting elements 218 to cause tissue injury.

The expandable mesh anchoring section 220 is located at the distal end of catheter body 214, having a similar structure to the expandable mesh section 202 of FIG. 13, with the exception of cutting elements 208. Thus, the expandable mesh anchoring section 220 expands at a desired location (e.g. a pulmonary vein 102), anchoring the cutting element deployment catheter 210 at a desired location.

The cutting element deployment arm 216 is positioned adjacent to catheter body 214, within an inner sheath 212, and can be advanced or retracted relative to the catheter body 214. As best seen in FIG. 15, cutting element deployment arm 216 contains longitudinally aligned cutting elements 218 with a driver rod 222 positioned proximal to the stack of cutting elements 214. The driver rod 222 may be advanced by the user from the control hub shown in FIG. 16 to push a cutting element 218 out of the cutting element deployment arm 216 and into the target tissue. To accomplish this, a simple threaded mechanism 230 as shown in FIG. 16 could be used. This thread 232 would advance the driver rod 222 by the length of one cutting element 218 with each rotation of the knob 235. The stack of cutting elements 218 is held in the end of the cutting element deployment arm 216 by a small elastically deflectable detent 237. This can only be pushed back by applying a significant force through the driver rod 222, pushing cutting element 218 past the detent 237 and out the end of the cutting element deployment arm 216. As this cutting element 218 passes by, the detent 237 springs back to block the passage of the next cutting element 218. This ensures that only one cutting element 214 is deployed at time.

In operation, a user advances the guide wire 114 to a desired location, such as a pulmonary vein 102, as seen in FIG. 14. Next, the catheter body 214 (within transseptal sheath 106) is advanced along the guide wire 114 until the distal end of the catheter body 214, i.e. the expandable mesh anchoring section 220, achieves a desired position, such as within a pulmonary vein 102. The inner sheath 212 is retracted, exposing the cutting element deployment arm 216. The user then advances the cutting element deployment arm 216 to the target tissue location, such as the ostium 100 of the pulmonary vein, and actuates the driver rod 222 to deploy a cutting element 218 into the tissue. The cutting element deployment arm may be repositioned at varying positions around the catheter body 214 to deploy cutting elements 218 at additional locations. When cutting element 218 deployment is complete, the user retracts the cutting element deployment arm 216, contracts the expandable mesh anchoring section 220, and removes the cutting element deployment catheter 210 from the patient. As with the devices described in FIGS. 9 and 10, these deployed cutting elements can be either permanent implants or made of biodegradable materials. They create a scarring healing response both to the mechanical cutting of their deployment and also as a response to the material left as an implant.

In yet another preferred embodiment according to this invention, a cutting element is coated with a drug or other material which would be deposited into the cuts made by the elements. In this embodiment the basic mechanism of scar generation changes from being purely a response to the mechanical injury and associated bleeding, to being a combination of the mechanical injury and the response to the drug or material. Some possible coatings for this embodiment would include glutaraldehyde, tetracycline, actinomycin, and polidocanol, ethanol, talc, or any other substance that induces scar formation. Moreover, the device may be hollow, with fluid pumped through the system to supply needed concentrations for scar induction all along the course of the device as it contacts tissue.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A device for causing tissue injury comprising:
a tube having a fixation element disposed at a distal end of said tube;
a deployment arm connected to said tube;
a plurality of cutting elements disposable within said deployment arm.
a mechanism for ejecting at least one cutting element to a target tissue site when said fixation element has retained said tube in a desired position.

2. A device as set forth in claim 1, wherein said fixation element is an expandable mesh.

3. A device as set forth in claim 1, wherein said device is a catheter assembly.

4. A device as set forth in claim 1, wherein said cutting element includes a coating to enhance tissue inflammation.

5. A device as set forth in claim 1, wherein said cutting element includes a coating to enhance scarring.

6. A device as set forth in claim 4, wherein said coating is selected from a group comprising glutaraldehyde, tetracycline, actinomicin, and polidocanol.
